(19) United States
(12) Patent Application Publication
(10) Pub. No.: US 2023/0415549 A1
(43) Pub. Date: Dec. 28, 2023

(54) INFORMATION PROCESSING APPARATUS, SYSTEM, AND METHOD

(71) Applicant: TOYOTA JIDOSHA KABUSHIKI KAISHA, Toyota-shi (JP)

(72) Inventor: Sachiko YOSHIDA, Koto-ku (JP)

(73) Assignee: TOYOTA JIDOSHA KABUSHIKI KAISHA, Toyota-shi (JP)

(21) Appl. No.: 18/334,542

(22) Filed: Jun. 14, 2023

(30) Foreign Application Priority Data
Jun. 22, 2022 (JP) 2022-100679

(51) Int. Cl. B60J 1/17; A61L 9/01; A61L 9/14; G08G 1/00

(52) U.S. Cl. CPC B60J 1/17; A61L 9/01; A61L 9/14; G08G 1/202; A61L 2209/111

(57) ABSTRACT

An information processing apparatus includes a controller. The controller is configured to control spraying of a cleaning agent in an interior of a vehicle after an end of use of the vehicle. The controller is configured to control a window of the vehicle to be open before a start of use of the vehicle.

### CROSS-REFERENCE TO RELATED APPLICATION

This application claims priority to Japanese Patent Application No. 2022-100679 filed on Jun. 22, 2022, the entire contents of which are incorporated herein by reference.

### TECHNICAL FIELD

The present disclosure relates to an information processing apparatus, a system, and a method.

### BACKGROUND

Technology for cleaning the interior of vehicles is known. For example, Patent Literature (PTL) 1 discloses an automatic deodorant control apparatus in a vehicle that is configured to control execution of deodorant operations by a deodorant apparatus.

### CITATION LIST

### Patent Literature

PTL 1: JP 2019-97933 A

### SUMMARY

There is room for improvement with respect to technology for cleaning the interior of vehicles.

It would be helpful to provide improved technology for cleaning the interior of vehicles.

An information processing apparatus according to an embodiment of the present disclosure includes a controller configured to:
control spraying of a cleaning agent in an interior of a vehicle after an end of use of the vehicle; and
control a window of the vehicle to be open before a start of use of the vehicle.

A system according to an embodiment of the present disclosure includes:
the information processing apparatus; and
the vehicle.

A method according to an embodiment of the present disclosure includes:
controlling spraying of a cleaning agent in an interior of a vehicle after an end of use of the vehicle; and
controlling a window of the vehicle to be open before a start of use of the vehicle.

According to an embodiment of the present disclosure, improved technology for cleaning the interior of vehicles can be provided.

### BRIEF DESCRIPTION OF THE DRAWINGS

In the accompanying drawings:

FIG. 1 is a diagram illustrating a schematic configuration of a system according to an embodiment of the present disclosure;

FIG. 2 is a block diagram of the system illustrated in FIG. 1;

FIG. 3 is a timing chart illustrating operations of the system illustrated in FIG. 1; and

FIG. 4 is a sequence diagram illustrating the operations of the system illustrated in FIG. 1.

### DETAILED DESCRIPTION

An embodiment of the present disclosure will be described below, with reference to the drawings.

(Configuration of System)

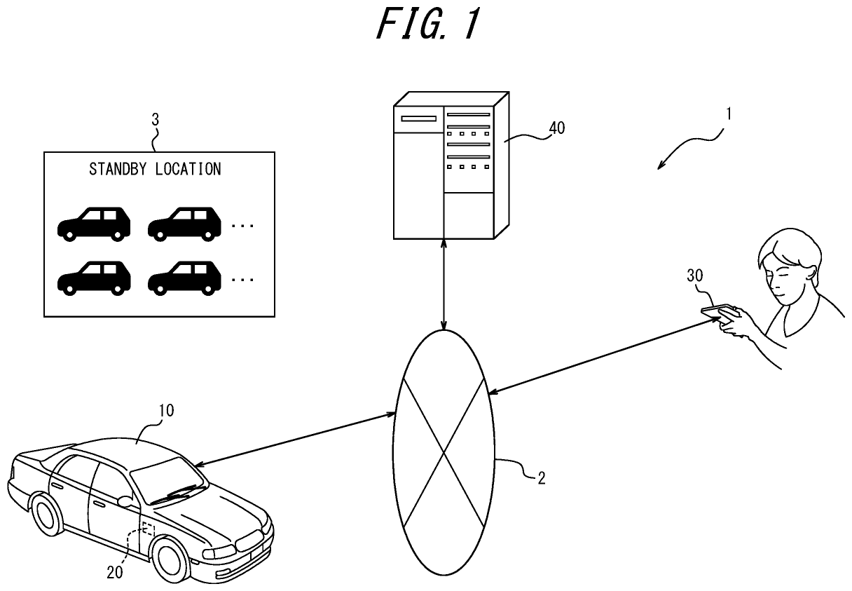

A system 1, as illustrated in FIG. 1, can provide car sharing service. The system 1 includes at least one vehicle 10, a terminal apparatus 30, and an information processing apparatus 40.

The vehicle 10, the terminal apparatus 30, and the information processing apparatus 40 are communicable via a network 2. The network 2 may be any network including a mobile communication network, the Internet, or the like.

The vehicle 10 can be used in the car sharing service. The vehicle 10 is, for example, a shared vehicle in the car sharing service. The vehicle 10 is used by users. The vehicle 10 waits at a standby location 3 when not in use by any user. The standby location 3 is, for example, any parking lot, such as a multi-level parking garage, underground parking garage, or outdoor parking garage.

The vehicle 10 may be any type of automobile. The vehicle 10 is, for example, a gasoline automobile, a Battery Electric Vehicle (BEV), a Hybrid Electric Vehicle (HEV), a Plug-in Hybrid Electric Vehicle (PHEV), a Fuel Cell Electric Vehicle (FCEV), or the like. The vehicle 10 may be a dedicated Mobility as a Service (MaaS) vehicle.

The vehicle 10 can travel automatically based on the control of the information processing apparatus 40. The vehicle 10 may be an Autonomous Vehicle (AV). The vehicle 10 travels automatically from the standby location 3 to a dispatch location designated by a user. After an end of use of the vehicle 10 by the user, the vehicle 10 returns to the standby location 3 by automatic traveling. The driving of the vehicle 10 may be automated at any level. The level of automation is, for example, one of level 1 to level 5 according to the classification of the Society of Automotive Engineers (SAE). However, the vehicle 10 may be driven by the user when being used by the user.

The vehicle 10 is equipped with a device 20. The device 20 is capable of spraying a cleaning agent. The cleaning agent includes, for example, a deodorizer and/or a sanitizer. However, the cleaning agent may include any agent. As another example, the cleaning agent may include a deodorizer, a sanitizer, and/or an air freshener.

The terminal apparatus 30 is used by the user. On the terminal apparatus 30, a dedicated application for using the car sharing service may be installed. The terminal apparatus 30 may be any appropriate apparatus, which can be connected to the network 2. The terminal apparatus 30 is, for example, a mobile phone, a smartphone, a tablet, a personal computer (PC), or the like.

The information processing apparatus 40 manages the vehicle 10. For example, the information processing apparatus 40 controls the vehicle 10 to travel automatically from the standby location 3 to the dispatch location designated by the user. After an end of use of the vehicle 10 by the user, the information processing apparatus 40 controls the vehicle 10 to travel automatically to the standby location 3. In addition, the information processing apparatus 40 controls the device 20 to spray the cleaning agent in an interior of the vehicle 10, after the end of use of the vehicle 10 by the user.

The information processing apparatus 40 may be operated by an entity providing the car sharing service. The information processing apparatus 40 is, for example, a dedicated computer configured to function as a server, a general purpose personal computer, a cloud computing system, or the like.

<Configuration of Vehicle>

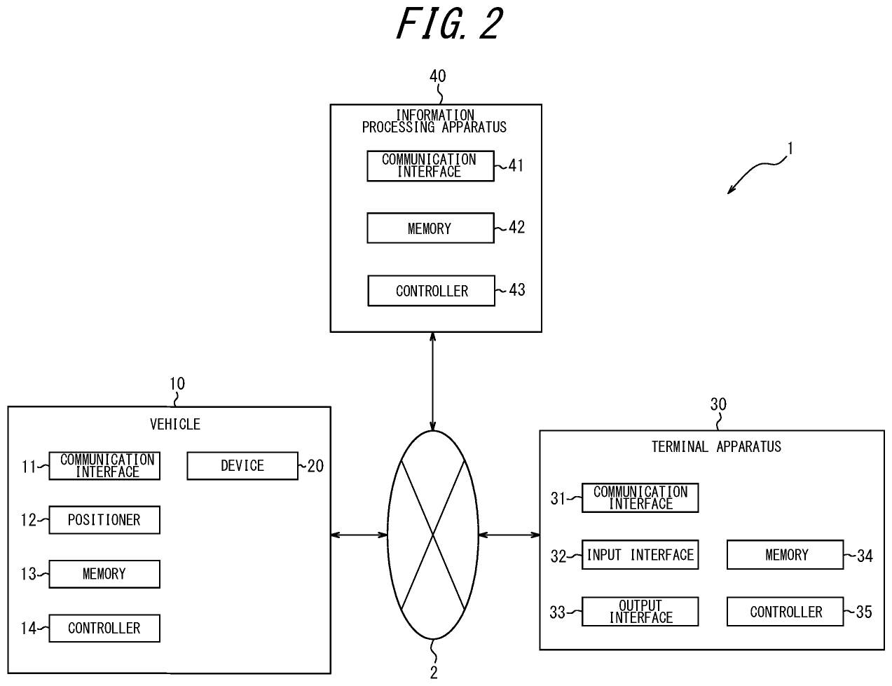

As illustrated in FIG. 2, the vehicle 10 includes a communication interface 11, a positioner 12, a memory 13, and a controller 14, in addition to the device 20.

The communication interface 11 is configured to include at least one communication module for connection to the network 2. The communication module is a communication module compliant with a mobile communication standard such as Long Term Evolution (LTE), 4th Generation (4G), or 5th Generation (5G). However, the communication module is not limited to this. The communication module may be compliant with any communication standard.

The positioner 12 is capable of acquiring positional information on the vehicle 10. The positioner 12 is configured to include at least one receiving module compliant with a satellite positioning system. The receiving module is, for example, a receiving module corresponding to the Global Positioning System (GPS). The receiving module is not, however, limited to this example. The receiving module may be a receiving module corresponding to any appropriate satellite positioning system.

The memory 13 is configured to include at least one semiconductor memory, at least one magnetic memory, at least one optical memory, or a combination of at least two of these. The semiconductor memory is, for example, random access memory (RAM) or read only memory (ROM). The RAM is, for example, static random access memory (SRAM), dynamic random access memory (DRAM), or the like. The ROM is, for example, electrically erasable programmable read only memory (EEPROM) or the like. The memory 13 may function as a main memory, an auxiliary memory, or a cache memory. The memory 13 stores data to be used for operations of the vehicle 10 and data obtained by the operations of the vehicle 10.

The controller 14 is configured to include at least one processor, at least one dedicated circuit, or a combination thereof. The processor is, for example, a general purpose processor such as a Central Processing Unit (CPU) or a Graphics Processing Unit (GPU), or a dedicated processor that is dedicated to a specific process. The dedicated circuit is, for example, a field-programmable gate array (FPGA), an application specific integrated circuit (ASIC), or the like. The controller 14 executes processes related to the operations of the vehicle 10 while controlling components of the vehicle 10.

The device 20 is capable of spraying a cleaning agent, as described above. In the present embodiment, the cleaning agent includes a deodorizer and a sanitizer. However, the cleaning agent may include any agent. The device 20 is disposed in an interior of the vehicle 10. The device 20 is located, in the interior of the vehicle 10, in such a position as to be able to deodorize and sanitize the interior of the vehicle 10 by spraying the deodorizer and the sanitizer.

The device 20 has an operation mode and a stop mode. In the operation mode, the device 20 sprays the deodorizer and the sanitizer. The deodorizer and sanitizer sprayed by the device 20 deodorize and sanitize the interior of the vehicle 10. In the stop mode, the device 20 stops spraying the deodorizer and the sanitizer.

The device 20 shifts into the operation mode or the stop mode based on the control of the controller 14. Here, the device 20 operates by receiving power supply from the vehicle 10. The power supply to the device 20 stops when an ignition of the vehicle 10 is turned off. Therefore, when the ignition of the vehicle 10 is turned off after the device 20 shifts into the operation mode, the device 20 shifts into the stop mode.

The functions of the vehicle 10 can be realized by execution of a vehicle program according to the present embodiment by a processor corresponding to the controller 14. That is, the functions of the vehicle 10 are realized by software. The vehicle program can cause a computer to function as the vehicle 10 by causing the computer to execute the operations of the vehicle 10. That is, the computer can function as the vehicle 10 by executing the operations of the vehicle 10 in accordance with the vehicle program.

Some or all of the functions of the vehicle 10 may be implemented by a dedicated circuit corresponding to the controller 14. That is, some or all of the functions of the vehicle 10 may be realized by hardware.

The controller 14 controls the positioner 12 to acquire positional information on the vehicle 10. The controller 14 controls the communication interface 11 to transmit the acquired positional information on the vehicle 10 to the information processing apparatus 40 via the network 2. The controller 14 may acquire, at established time intervals, the positional information on the vehicle 10 and transmit the acquired positional information on the vehicle 10 to the information processing apparatus 40. The time intervals may be set based on an average speed or the like of the vehicle 10.

<Configuration of Terminal Apparatus>

As illustrated in FIG. 2, the terminal apparatus 30 includes a communication interface 31, an input interface 32, an output interface 33, a memory 34, and a controller 35.

The communication interface 31 is configured to include at least one communication module for connection to the network 2. The communication module is, for example, a communication module compliant with a mobile communication standard such as LTE, 4G, or 5G. However, the communication module is not limited to this. The communication module may be compliant with any communication standard.

The input interface 32 is capable of accepting an input from a user. The input interface 32 is configured to include at least one interface for input that is capable of accepting an input from a user. The interface for input is, for example, a physical key, a capacitive key, a pointing device, a touch screen integrally provided with a display, a microphone, or the like. However, the interface for input is not limited to this.

The output interface 33 is capable of outputting data. The output interface 33 is configured to include at least one interface for output that is capable of outputting data. The interface for output is, for example, a display, a speaker, or the like. The display is, for example, a liquid crystal display (LCD), an organic electro-luminescent (EL) display, or the like. However, the interface for output is not limited to this.

The memory 34 is configured to include at least one semiconductor memory, at least one magnetic memory, at least one optical memory, or a combination of at least two of these. The semiconductor memory is, for example, RAM, ROM, or the like. The RAM is, for example, SRAM, DRAM, or the like. The ROM is, for example, EEPROM or the like. The memory 34 may function as a main memory, an auxiliary memory, a cache memory, or the like. The memory 34 stores data to be used for operations of the terminal apparatus 30 and data obtained by the operations of the terminal apparatus 30.

The controller 35 is configured to include at least one processor, at least one dedicated circuit, or a combination thereof. The processor is, for example, a general purpose processor such as a CPU or a GPU, or a dedicated processor that is dedicated to a specific process. The dedicated circuit is, for example, an FPGA, an ASIC, or the like. The controller 35 executes processes related to the operations of the terminal apparatus 30 while controlling components of the terminal apparatus 30.

The functions of the terminal apparatus 30 are realized by execution of a terminal program according to the present embodiment by a processor corresponding to the controller 35. That is, the functions of the terminal apparatus 30 are realized by software. The terminal program causes a computer to execute the operations of the terminal apparatus 30, thereby causing the computer to function as the terminal apparatus 30. That is, the computer executes the operations of the terminal apparatus 30 in accordance with the terminal program to thereby function as the terminal apparatus 30.

Some or all of the functions of the terminal apparatus 30 may be implemented by a dedicated circuit corresponding to the controller 35. That is, some or all of the functions of the terminal apparatus 30 may be realized by hardware.

<Configuration of Information Processing Apparatus>

As illustrated in FIG. 2, the information processing apparatus 40 includes a communication interface 41, a memory 42, and a controller 43.

The communication interface 41 is configured to include at least one communication module for connection to the network 2. For example, the communication module is a communication module compliant with a standard such as a wired Local Area Network (LAN) or a wireless LAN. However, the communication module is not limited to this. The communication module may be compliant with any communication standard. The communication interface 41 is connectable to the network 2 via a wired LAN or a wireless LAN using the communication module.

The memory 42 is configured to include at least one semiconductor memory, at least one magnetic memory, at least one optical memory, or a combination of at least two of these. The semiconductor memory is, for example, RAM, ROM, or the like. The RAM is, for example, SRAM, DRAM, or the like. The ROM is, for example, EEPROM or the like. The memory 42 may function as a main memory, an auxiliary memory, or a cache memory. The memory 42 stores data to be used for operations of the information processing apparatus 40 and data obtained by the operations of the information processing apparatus 40. For example, the memory 42 stores map information.

The controller 43 is configured to include at least one processor, at least one dedicated circuit, or a combination thereof. The processor is, for example, a general purpose processor such as a CPU or a GPU, or a dedicated processor that is dedicated to a specific process. The dedicated circuit is, for example, an FPGA, an ASIC, or the like. The controller 43 executes processes related to the operations of the information processing apparatus 40 while controlling components of the information processing apparatus 40.

The functions of the information processing apparatus 40 may be implemented by a processor corresponding to the controller 43 executing a processing program according to the present embodiment. That is, the functions of the information processing apparatus 40 may be implemented by software. The processing program enables a computer to function as the information processing apparatus 40 by causing the computer to execute the operations of the information processing apparatus 40. That is, the computer functions as the information processing apparatus 40 by executing the operations of the information processing apparatus 40 in accordance with the processing program.

Some or all of the functions of the information processing apparatus 40 may be realized by a dedicated circuit corresponding to the controller 43. That is, some or all of the functions of the information processing apparatus 40 may be realized by hardware.

(System Processes)

The processes by the system 1 are described below.

<Car Dispatch Process>

When a user wants to use the vehicle 10, the user enters, to the input interface 32 of the terminal apparatus 30, an input commanding dispatch of the vehicle 10 and an input indicating a dispatch location that the user designates. In the terminal apparatus 30, the controller 35 accepts the inputs through the input interface 32. Upon accepting the inputs, the controller 35 transmits, through the communication interface 31, a dispatch command signal commanding dispatch of the vehicle 10 and information on the dispatch location to the information processing apparatus 40 via the network 2.

In the information processing apparatus 40, the controller 43 receives, through the communication interface 41, the dispatch command signal and the information on the dispatch location from the terminal apparatus 30 via the network 2. Upon receiving the dispatch command signal and the like, the controller 43 controls the vehicle 10 to travel automatically from the standby location 3 to the dispatch location. As an example of the control, the controller 43 receives, through the communication interface 41, positional information on the vehicle 10 from the vehicle 10 via the network 2. The controller 43 generates, based on the received positional information on the vehicle 10 and map information in the memory 42, a control signal to control the vehicle 10 to travel automatically. The controller 43 transmits, through the communication interface 41, the generated control signal to the vehicle 10 via the network 2. In the vehicle 10, the controller 14 receives, through the communication interface 11, the control signal from the information processing apparatus 40 via the network 2. The controller 14 executes automatic traveling of the vehicle 10 based on the received control signal.

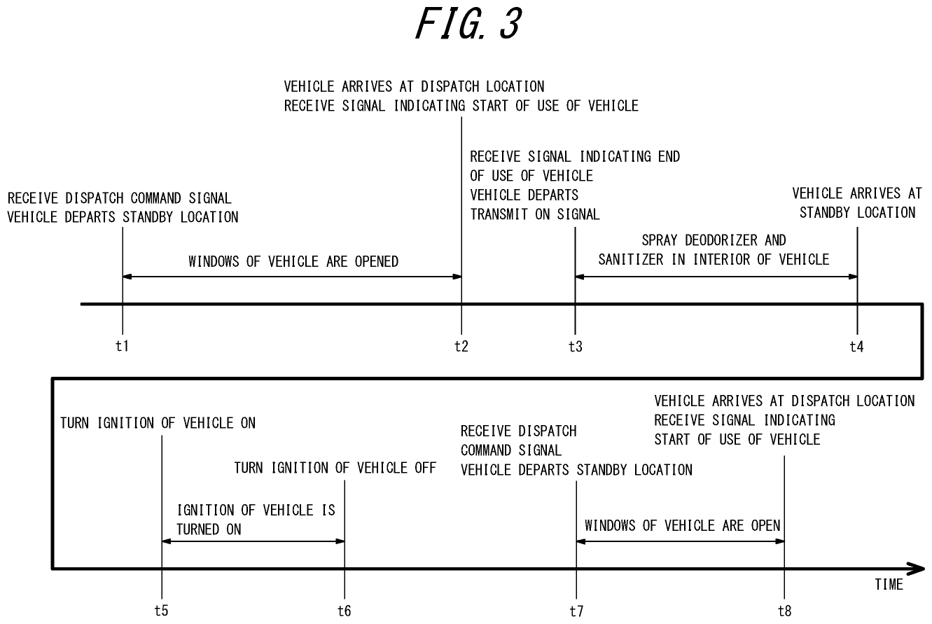

For example, in FIG. 3, at a time t1, the information processing apparatus 40 receives the dispatch command signal from the terminal apparatus 30. At the time t1, the vehicle 10 departs the standby location 3 based on the control of the information processing apparatus 40. At a time t2, the vehicle 10 arrives at the dispatch location based on the control of the information processing apparatus 40. From the time t1 to the time t2, the vehicle 10 travels automatically based on the control of the information processing apparatus 40.

As the vehicle 10 travels automatically from the standby location 3 to the dispatch location, the vehicle 10 is automatically dispatched to the dispatch location. The automatic dispatch of the vehicle 10 to the dispatch location eliminates the need for the user to go to the standby location 3. This configuration improves convenience of the user.

The user starts using the vehicle 10 having been dispatched to the dispatch location. When starting use of the vehicle 10, the user enters, into the input interface 32 of the terminal apparatus 30, an input indicating a start of use of the vehicle 10. In the terminal apparatus 30, the controller 35 accepts the input through the input interface 32. Upon receiving the input, the controller 35 transmits, through the communication interface 31, a signal indicating a start of use of the vehicle 10 to the information processing apparatus 40 via the network 2. In the information processing apparatus 40, the controller 43 receives, through the communication interface 41, the signal indicating the start of use of the vehicle 10 from the terminal apparatus 30 via the network 2. For example, in FIG. 3, at the time t2, the controller 43 receives the signal indicating the start of use of the vehicle 10 from the terminal apparatus 30.

<Return Process>

When ending use of the vehicle 10, the user enters, into the input interface 32 of the terminal apparatus 30, an input indicating an end of use of the vehicle 10. In the terminal apparatus 30, the controller 35 accepts the input through the input interface 32. Upon receiving the input, the controller 35 transmits, through the communication interface 31, a signal indicating an end of use of the vehicle 10 to the information processing apparatus 40 via the network 2.

In the information processing apparatus 40, the controller 43 receives, through the communication interface 41, the signal indicating the end of use of the vehicle 10 from the terminal apparatus 30 via the network 2. Upon receiving the signal indicating the end of use of the vehicle 10, the controller 43 controls the vehicle 10 to travel automatically from a current position to the standby location 3. The controller 43 may control the vehicle 10 to travel automatically by generating a control signal and transmitting the generated control signal to the vehicle 10, in the same or similar manner as the dispatch process.

For example, in FIG. 3, at a time t3, the information processing apparatus 40 receives, from the terminal apparatus 30, the signal indicating the end of use of the vehicle 10. At the time t3, the vehicle departs for the standby location 3 based on the control of the information processing apparatus 40. At a time t4, the vehicle 10 arrives at the standby location 3 based on the control of the information processing apparatus 40. From the time t3 to the time t4, the vehicle 10 travels automatically based on the control of the information processing apparatus 40.

In this way, the vehicle 10 travels automatically to the standby location 3, so the vehicle 10 is automatically returned to the standby location 3. The automatic return of the vehicle 10 eliminates the user having to return the vehicle 10 to the standby location 3. This configuration improves convenience of the user.

<Deodorization and Sterilization Process>

In the information processing apparatus 40, the controller 43 controls spraying of a cleaning agent in an interior of the vehicle 10 after the end of use of the vehicle 10. In the present embodiment, the controller 43 controls spraying of a deodorizer and a sanitizer in the interior of the vehicle 10. Spraying the deodorizer and the sanitizer in the interior of the vehicle 10, after the end of use of the vehicle 10, allows for quick deodorization and sanitization of the interior of the vehicle 10.

In the present embodiment, the controller 43 controls the device 20 to shift into the operation mode, so that the deodorizer and the sanitizer are sprayed in the interior of the vehicle 10. The controller 43 controls the device 20 to shift into the operation mode by transmitting, through the communication interface 41, an ON signal to the vehicle 10 via the network 2. The ON signal is a control signal to shift the device 20 into the operation mode. In the vehicle 10, the controller 14 shifts the device 20 into the operation mode upon receiving, through the communication interface 11, the ON signal from the information processing apparatus 40 via the network 2.

The controller 43 may control the device 20 to shift into the operation mode when the vehicle 10 returns to the standby location 3 after the end of use of the vehicle 10. The controller 43 may control the device 20 to shift into the operation mode upon receiving, from the terminal apparatus 30, the signal indicating the end of use of the vehicle 10. For example, in FIG. 3, upon receiving the signal indicating the end of use of the vehicle 10 at the time t3, the controller 43 may transmit the ON signal to the vehicle 10 at the time t3. This configuration allows for quick deodorization and sterilization of the interior of the vehicle 10.

The controller 43 may control the device 20 to remain in the operation mode while the vehicle 10 is traveling automatically to the standby location 3 after the end of use of the vehicle 10. Here, as described above, the device 20 operates by receiving power supply from the vehicle 10. Therefore, when the vehicle 10 arrives at the standby location 3 and the ignition of the vehicle 10 is turned off, the device 20 shifts into the stop mode. In other words, the controller 43 can control the device 20 to remain in the operation mode, while the vehicle 10 is traveling automatically to the standby location 3, by not transmitting an OFF signal to the vehicle 10 after transmitting the ON signal to the vehicle 10 as described above.

For example, in FIG. 3, the controller 43 may transmit the ON signal to the vehicle 10 at the time t3. By transmitting the ON signal to the vehicle 10 at the time t3, the device 20 can be put into the operation mode. At the time t4, the vehicle 10 arrives at the standby location 3, as described above, and the ignition of the vehicle 10 is turned off. Upon turning off the ignition of the vehicle 10 at the time t4, the device 20 shifts into the stop mode. This configuration allows the device 20 to remain in the operation mode from the time t3 to the time t4 while the vehicle 10 is traveling automatically.

Thus, by deodorizing and sanitizing the interior of the vehicle 10 while the vehicle 10 is traveling automatically, the interior of the vehicle 10 can be deodorized and sanitized when the vehicle 10 is likely to be unattended.

Here, the controller 43 may control the ignition of the vehicle 10 to remain on until at least first time elapses after the end of use of the vehicle 10. The first time may be set based on time it takes to deodorize and sanitize the interior of the vehicle. Upon receiving the signal indicating the end of use of the vehicle 10, the controller 43 may transmit, through the communication interface 41, a maintenance signal to the vehicle 10 via the network 2. The maintenance signal is a control signal to keep the ignition of the vehicle 10 on until the first time elapses. In the vehicle 10, the controller 14 controls the ignition of the vehicle 10 to remain on until the first time elapses upon receiving, by the communication interface 11, the maintenance signal from the information processing apparatus 40 via the network 2. In other words, the controller 14 controls the ignition of the vehicle 10 to remain on until the first time elapses, even in a case in which the vehicle 10 arrives at the standby location 3 before the first time elapses.

For example, in FIG. 3, the controller 43 may transmit the maintenance signal along with the ON signal to the vehicle 10 at the time t3. Here, depending on a location at which the user has ended the use of the vehicle 10, time from the time t3 to the time t4 while the vehicle 10 is traveling automatically may not be long enough to perform deodorization and sanitization of the interior of the vehicle 10. In this case, if the device 20 shifts into the stop mode by turning off the ignition of vehicle 10 when the vehicle 10 arrives at the standby location 3 at the time t4, it is assumed that the device 20 cannot sufficiently deodorize and sanitize the interior of the vehicle 10. Even in such a case, the ignition of the vehicle 10 remains on until at least the first time elapses after the end of use of the vehicle 10, thereby ensuring power supply to maintain the device 20 in the operation mode.

The controller 43 may also control the device 20 to shift into the stop mode at a point in time when second time has elapsed after the shift of the device 20 into the operation mode. The second time may be set based on time required to deodorize and sanitize the interior of the vehicle. The second time may be the same length as the first time or different from the first time. In the present embodiment, the controller 43 controls the device 20 to shift into the stop mode by transmitting, through the communication interface 41, an OFF signal to the vehicle 10 via the network 2. The OFF signal is a control signal to shift the device into the stop mode. In the vehicle 10, the controller 14 shifts the device 20 into the stop mode upon receiving, by the communication interface 11, the OFF signal from the information processing apparatus via the network 2. The controller 43 may transmit the OFF signal to the vehicle 10 at a point in time when the second time has elapsed after the transmission of the ON signal to the vehicle 10.

For example, in FIG. 3, the controller 43 may transmit the OFF signal to the vehicle 10 at a point in time when the second time has elapsed after the transmission of the ON signal at the time t3. Here, depending on the location at which the user has ended the use of the vehicle 10, time from the time t3 to the time t4 while the vehicle 10 is traveling automatically may be longer than time required to deodorize and sanitize the interior of the vehicle 10. In such a case, the device 20 is put into the stop mode at a point in time when the second time has elapsed after the shift of the device 20 into the operation mode, so the deodorizer and sanitizer in the device 20 can be used as appropriate.

<Window Opening/Closing Process>

In the information processing apparatus 40, the controller 43 controls windows of the vehicle 10 to be open before the start of use of the vehicle 10. By controlling the windows of the vehicle 10 to be open before the start of use of the vehicle 10, residues of the deodorizer and sanitizer in the interior of the vehicle 10 can be discharged outside the vehicle 10. This configuration allows the user to use the vehicle 10 without feeling uncomfortable with smell of the deodorizer or other odors.

In the present embodiment, the controller 43 controls the windows of the vehicle 10 to open by transmitting, through the communication interface 41, an open signal to the vehicle 10 via the network 2. The open signal is a control signal to control the windows of the vehicle 10 to open. In a case in which the vehicle 10 has multiple windows, the open signal may specify which of the multiple windows of the vehicle 10 to open. The open signal may specify an amount of opening of the windows of the vehicle 10. The amount of opening of the windows of the vehicle 10 may be set to account for theft that may occur at the vehicle 10. The amount of opening of the windows of the vehicle 10 may be such that a person cannot put a hand from the windows. In the vehicle 10, upon receiving, by the communication interface 11, the open signal from the information processing apparatus 40 through the network 2, the controller 14 controls the windows of the vehicle 10 to open based on the open signal.

The controller 43 may determine the amount of opening of the windows of the vehicle 10 based on weather information. The open signal may specify the determined amount of opening of the windows of the vehicle 10. The controller 43 may receive, through the communication interface 41, weather information for an area containing the standby location 3 and the dispatch location from an external server via the network 2. The controller 43 may determine to open a more amount of opening of the windows in the vehicle 10 when weather in the area containing the standby location 3 and the dispatch location is sunny than when it is rainy. This configuration allows for efficient ventilation of the interior of the vehicle 10 when the weather is sunny. The controller 43 may also determine to open a less amount of opening of the windows in the vehicle 10 when weather in the area containing the standby location 3 and the dispatch location is rainy than when it is sunny. This configuration can prevent rain from blowing into the interior of the vehicle 10 when the weather is rainy.

In the present embodiment, the controller 43 controls the windows of the vehicle 10 to close by transmitting, through the communication interface 41, a close signal to the vehicle 10 via the network 2. The close signal is a control signal to control the windows of the vehicle 10 to close. In a case in which the vehicle 10 has multiple windows, the close signal may specify which of the multiple windows of the vehicle 10 to close. In the vehicle 10, upon receiving, by the communication interface 11, the close signal from the information processing apparatus 40 through the network 2, the controller 14 controls the windows of the vehicle 10 to close based on the close signal.

The controller 43 may control the windows of the vehicle 10 to be open while the vehicle 10 is traveling automatically from the standby location 3 to the dispatch location before the start of use of the vehicle 10.

For example, in FIG. 3, the controller 43 may transmit the open signal to the vehicle 10 at the time t1 and the close signal to the vehicle 10 at the time t2. With this configuration, the windows of the vehicle 10 are open from the time t1 to the time t2 while the vehicle 10 is traveling automatically from the standby location 3 to the dispatch location.

Thus, since the windows of the vehicle 10 are open while the vehicle 10 is traveling automatically, the possibility of a stranger entering the vehicle 10 through a window of the vehicle 10 is reduced. This configuration reduces the possibility of theft occurring at the vehicle 10, even if the vehicle 10 is unattended.

The controller 43 may control the windows of the vehicle 10 to open when the vehicle 10 departs from the standby location 3. For example, in FIG. 3, the controller 43 may transmit the open signal to the vehicle 10 at the time t1 when the vehicle 10 departs from the standby location 3.

The controller 43 may control the windows of the vehicle 10 to

open upon receiving, by the communication interface 41, the dispatch command signal from the terminal apparatus 30 via the network 2. For example, in FIG. 3, the controller 43 may transmit the open signal to the vehicle 10 at the time t1 of receiving the dispatch command signal.

The controller 43 may control the windows of the vehicle 10 to close before the vehicle 10 arrives at the dispatch location. For example, in FIG. 3, the controller 43 may transmit the close signal to the vehicle 10 third time before the time t2, after the transmission of the open signal at the time t1. The third time may be set in consideration of time it takes to discharge the deodorizer and sanitizer remaining in the interior of the vehicle to the outside. The windows of the vehicle 10 are closed before the vehicle 10 arrives at the dispatch location, so that the windows of the vehicle 10 are closed before the vehicle 10 stops at the dispatch location. This configuration reduces the possibility of theft occurring in the vehicle 10.

The controller 43 may control the windows of the vehicle 10 to close upon receiving, by the communication interface 41, the signal indicating the start of use of the vehicle 10 from the terminal apparatus via the network 2. For example, in FIG. 3, the controller 43 may transmit the close signal to the vehicle 10 at the time t2 at which the signal indicating the start of use of the vehicle 10 is received, after the transmission of the open signal at the time t1.

In a case in which the vehicle 10 waits at the standby location 3 before the start of use of the vehicle 10, the controller 43 may control the windows of the vehicle 10 to be open when the ignition of the vehicle 10 is turned on at the standby location 3.

For example, in FIG. 3, the vehicle 10 is assumed to be a BEV. It is also assumed that charging of the vehicle 10 is performed at a charging facility at the standby location 3. The controller 43 controls automatic traveling of the vehicle 10 so that the vehicle 10 travels to the charging facility between a time t5 and a time t6. At the time t5, the controller 43 controls the ignition of the vehicle 10 to be turned on and then transmits the open signal to the vehicle 10. At the time t6, the controller 43 transmits the close signal to the vehicle 10 and then controls the ignition of the vehicle 10 to be turned off. This configuration allows the windows of the vehicle 10 to be open from the time t5 to the time t6, when the ignition of the vehicle 10 is turned on.

After this, at a time t7, similar to the time t1, the information processing apparatus 40 receives a dispatch command signal from the terminal apparatus 30. At the time t7, the vehicle 10 departs from the standby location 3 under the control of the information processing apparatus 40. At a time t8, similar to the time t2, the vehicle 10 arrives at a dispatch location under the control of the information processing apparatus 40. At the time t8, the information processing apparatus 40 receives, from the terminal apparatus 30, a signal indicating a start of use of the vehicle 10. In other words, from the time t7 to the time t8, which is before the start of use of the vehicle 10, the windows of the vehicle 10 are open. Here, depending on the dispatch location designated by a user, time from the time t7 to the time t8 may not be long enough for the deodorizer and sanitizer remaining in the interior of the vehicle 10 to be discharged out of the vehicle 10 through the windows. Even in such a case, when the ignition of the vehicle 10 is turned on at the standby location 3, such as between the time t5 and the time t6, the windows of the vehicle 10 are opened to ensure that the deodorizer and sanitizer remaining in the interior of the vehicle 10 can be discharged out of the vehicle 10.

Depending on the timing of the control, the close signal may not be transmitted from the information processing apparatus 40 to the vehicle 10 before the ignition of the vehicle 10 is turned off, after the vehicle 10 receives the open signal and opens the windows of the vehicle 10. Thus, in the vehicle 10, the controller 14 may maintain power to a mechanism that closes the windows of the vehicle 10, in a case in which the ignition of the vehicle 10 is turned off after opening the windows of the vehicle 10 and before receiving a close signal. In this case, after the controller 14 receives the close signal and closes the windows of the vehicle 10, the controller 14 may stop supplying power to the mechanism that closes the windows of the vehicle 10. Alternatively, the controller 14 may keep the ignition of the vehicle 10 on until the close signal is received, even after the vehicle 10 arrives at the standby location 3, for example. In this case, the controller 14 may turn off the ignition of the vehicle 10 after receiving the close signal and closing the windows of vehicle 10.

<Other Examples of Deodorization and Sanitization Process>

In the present embodiment, the controller 43 may control the deodorizer and the sanitizer to be sprayed in the interior of the vehicle 10 after an end of use of the vehicle 10. In other words, the controller 43 may control spraying of the deodorizer and the sanitizer in the interior of the vehicle 10, after an end of use of the vehicle 10 and before a start of next use of the vehicle 10. As an example, the controller 43 may control spraying of the deodorizer and the sanitizer in the interior of the vehicle 10 and control the windows of the vehicle 10 to be open, while the vehicle 10 is traveling automatically from the standby location 3 to a dispatch location before a start of use of the vehicle 10. For example, in FIG. 3, after the end of use of the vehicle 10 at the time t3, the use of the vehicle 10 is started at the time t8. The controller 43 may put the device 20 in the operation mode and control the windows of the to vehicle 10 to be open, between the time t7 and the time t8. The controller 43 may put the device 20 in the operation mode between the time t1 and the time t2, and further put the device 20 in the operation mode between the time t7 and the time t8.

(Operations of System)

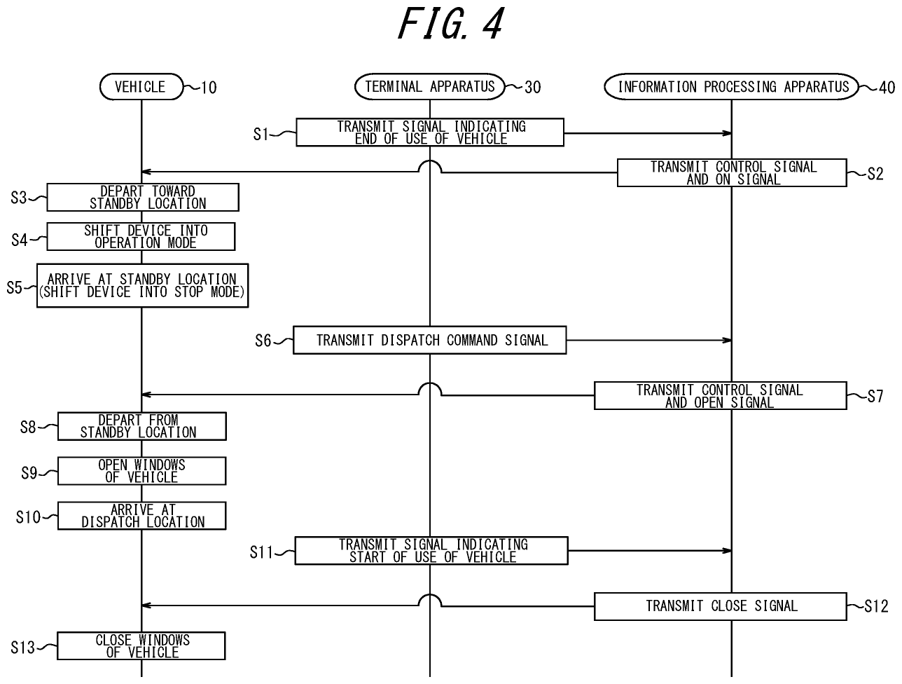

FIG. 4 is a sequence diagram illustrating operations of the system illustrated in FIG. 1. In the following process, the vehicle 10, the terminal apparatus 30, and the information processing apparatus 40 transmit and receive signals via the network 2.

In the process of step S1, the terminal apparatus 30 transmits, to the information processing apparatus 40, a signal indicating an end of use of the vehicle 10. The information processing apparatus 40 receives, from the terminal apparatus 30, the signal indicating the end of use of vehicle 10.

In the process of step S2, the information processing apparatus transmits, to the vehicle 10, a control signal for automatic traveling of the vehicle 10 and an ON signal. The vehicle 10 receives the control signal and the ON signal from the information processing apparatus 40.

In the process of step S3, the vehicle 10 departs from a current position toward the standby location 3 based on the control signal received from the information processing apparatus 40. In the process of step S4, the vehicle 10 shifts the device 20 into the operation mode based on the ON signal received from the information processing apparatus 40.

In the process of step S5, the vehicle 10 arrives at the standby location 3 based on the control signal received from the information processing apparatus 40. The ignition of the vehicle 10 is turned off as the vehicle 10 arrives at the standby location 3. When the ignition of the vehicle 10 is turned off, the device 20 shifts into the stop mode.

In the process of step S6, the terminal apparatus 30 transmits a dispatch command signal to the information processing apparatus 40. The information processing apparatus 40 receives the dispatch command signal from the terminal apparatus 30.

In the process of step S7, the information processing apparatus 40 transmits, to the vehicle 10, a control signal for controlling the vehicle 10 to travel automatically and an open signal. The vehicle 10 receives the control signal and the open signal from the information processing apparatus 40.

In the process of step S8, the vehicle 10 departs from the standby location 3 based on the control signal received from the information processing apparatus 40. In the process of step S9, the vehicle 10 opens the windows of the vehicle 10 based on the open signal received from the information processing apparatus 40.

In the process of step S10, the vehicle 10 arrives at a dispatch location based on the control signal received from the information processing apparatus 40.

In the process of step S11, the terminal apparatus 30 transmits, to the information processing apparatus 40, a signal indicating a start of use of the vehicle 10. The information processing apparatus 40 receives, from the terminal apparatus 30, the signal indicating the start of use of the vehicle 10.

In the process of step S12, the information processing apparatus 40 transmits a close signal to the vehicle 10. The vehicle 10 receives the close signal from the information processing apparatus 40.

In the process of step S13, the vehicle 10 closes the windows of the vehicle 10 based on the close signal received from the information processing apparatus 40.

As described above, in the information processing apparatus 40 according to the present embodiment, the controller 43 controls spraying of the deodorizer and the sanitizer in the interior of the vehicle 10 after an end of use of the vehicle 10. The deodorizer and sanitizer are sprayed in the interior of the vehicle 10 after the end of use of the vehicle 10, thereby quickly deodorizing and sanitizing the interior of the vehicle 10. In addition, the controller 43 controls the windows of the vehicle 10 to be open before a start of use of the vehicle 10. Residues of the deodorizer and sanitizer in the interior of the vehicle 10 are to some extent discharged out of the vehicle 10 by natural ventilation of the interior of the vehicle 10. In the present embodiment, the windows of the vehicle 10 are open before the start of use of the vehicle 10, thereby ensuring that the residues of the deodorizer and the sanitizer in the interior of the vehicle 10 can be more reliably discharged outside the vehicle 10. This configuration allows the user to use the vehicle 10 without feeling uncomfortable with smell of the deodorizer or other odors.

In addition, the controller 43 controls spraying of the deodorizer and the sanitizer in the interior of the vehicle 10 after the end of use of the vehicle 10, and controls the windows of the vehicle 10 to be open before the start of use of the vehicle 10. In other words, the controller 43 can clean the vehicle 10 according to the use of the vehicle 10. This configuration allows the vehicle 10 to be cleaned appropriately.

Thus, the present embodiment can provide improved technology for cleaning the vehicle 10.

While the present disclosure has been described with reference to the drawings and examples, it should be noted that various modifications and revisions may be implemented by those skilled in the art based on the present disclosure. Accordingly, such modifications and revisions are included within the scope of the present disclosure. For example, functions or the like included in each component, each step, or the like can be rearranged without logical inconsistency, and a plurality of components, steps, or the like can be combined into one or divided.

For example, the vehicle 10 may perform, as an information processing apparatus, the processing of the information processing apparatus 40 according to the embodiment described above. As an example, the vehicle 10, as an information processing apparatus, may control spraying of the deodorizer and the sanitizer in the interior of the vehicle 10 after an end of use of the vehicle 10, and control the windows of the vehicle 10 to be open before a start of use of the vehicle 10.

Examples of some embodiments of the present disclosure are described below. However, it should be noted that the embodiments of the present disclosure are not limited to these examples.

[Appendix 1] An information processing apparatus comprising a controller configured to:
control spraying of a cleaning agent in an interior of a vehicle after an end of use of the vehicle; and
control a window of the vehicle to be open before a start of use of the vehicle.
   [Appendix 2] The information processing apparatus according to appendix 1, wherein the controller is configured to control spraying of the cleaning agent in the interior of the vehicle by controlling a device capable of spraying the cleaning agent to shift into an operation mode.
   [Appendix 3] The information processing apparatus according to appendix 1 or 2, wherein the controller is configured to control the device to shift into the operation mode at a time when the vehicle returns to a standby location after the end of use of the vehicle.
   [Appendix 4] The information processing apparatus according to any one of appendices 1 to 3, further comprising a communication interface,

wherein the controller controls the device to shift into the operation mode upon receiving, by the communication interface, a signal indicating an end of use of the vehicle from a terminal apparatus of a user using the vehicle.

[Appendix 5] The information processing apparatus according to any one of appendices 1 to 4, wherein the controller controls the vehicle to travel automatically to a standby location upon receiving a signal indicating an end of use of the vehicle.
[Appendix 6] The information processing apparatus according to any one of appendices 1 to 5, wherein the controller is configured to control the device to maintain the operation mode while the vehicle is traveling automatically to a standby location after the end of use of the vehicle.
[Appendix 7] The information processing apparatus according to any one of appendices 1 to 6, wherein the controller is configured to control an ignition of the vehicle to remain on until at least first time elapses after the end of use of the vehicle.
[Appendix 8] The information processing apparatus according to any one of appendices 1 to 7, wherein the controller is configured to control the device to shift into a stop mode at a point in time when second time has elapsed after the shift of the device into the operation mode.
[Appendix 9] The information processing apparatus according to any one of appendices 1 to 8, wherein the controller is configured to control the window of the vehicle to be open while the vehicle is traveling automatically from a standby location to a dispatch location before the start of use of the vehicle.
[Appendix 10] The information processing apparatus according to any one of appendices 1 to 9, wherein the controller is configured to control the window of the vehicle to open when the vehicle departs a standby location.
[Appendix 11] The information processing apparatus according to any one of appendices 1 to 10, wherein the controller is configured to control the window of the vehicle to close before the vehicle arrives at a dispatch location.
[Appendix 12] The information processing apparatus according to any one of appendices 1 to 11, further comprising a communication interface,

wherein the controller controls the window of the vehicle to open upon receiving from a terminal apparatus of a user, by the communication interface, a dispatch command signal commanding dispatch of the vehicle.

[Appendix 13] The information processing apparatus according to any one of appendices 1 to 12, wherein the controller controls the window of the vehicle to close upon receiving from the terminal apparatus, by the communication interface, a signal indicating a start of use of the vehicle.
[Appendix 14] The information processing apparatus according to any one of appendices 1 to 13, wherein the controller controls the vehicle to travel automatically from a standby location to a dispatch location upon receiving, by the communication interface, the dispatch command signal.
[Appendix 15] The information processing apparatus according to any one of appendices 1 to 14, wherein in a case in which the vehicle waits at a standby location before the start of use of the vehicle, the controller controls the window of the vehicle to be open when an ignition of the vehicle is turned on at the standby location.
[Appendix 16] The information processing apparatus according to any one of appendices 1 to 15, wherein the controller is configured to determine an amount of opening of the window of the vehicle based on weather information.
[Appendix 17] The information processing apparatus according to any one of appendices 1 to 16, wherein the controller is configured to control spraying of the cleaning agent and control the window of the vehicle to be open while the vehicle is traveling automatically from a standby location to a dispatch location before the start of use of the vehicle.
[Appendix 18] The information processing apparatus according to any one of appendices 1 to 17, wherein the cleaning agent includes a deodorizer and/or a sanitizer.
[Appendix 19] A system comprising:

the information processing apparatus according to any one of appendices 1 to 18; and

the vehicle.

[Appendix 20] A method comprising:

controlling spraying of a cleaning agent in an interior of a vehicle after an end of use of the vehicle; and

controlling a window of the vehicle to be open before a start of use of the vehicle.

1. An information processing apparatus comprising a controller configured to:
control spraying of a cleaning agent in an interior of a vehicle after an end of use of the vehicle; and
control a window of the vehicle to be open before a start of use of the vehicle.

2. The information processing apparatus according to claim 1, wherein the controller is configured to control spraying of the cleaning agent in the interior of the vehicle by controlling a device capable of spraying the cleaning agent to shift into an operation mode.

3. The information processing apparatus according to claim 2, wherein the controller is configured to control the device to shift into the operation mode at a time when the vehicle returns to a standby location after the end of use of the vehicle.

4. The information processing apparatus according to claim 3, further comprising a communication interface,
wherein the controller controls the device to shift into the operation mode upon receiving, by the communication interface, a signal indicating an end of use of the vehicle from a terminal apparatus of a user using the vehicle.

5. The information processing apparatus according to claim 4, wherein the controller controls the vehicle to travel automatically to a standby location upon receiving the signal indicating an end of use of the vehicle.

6. The information processing apparatus according to claim 5, wherein the controller is configured to control the device to maintain the operation mode while the vehicle is traveling automatically to the standby location after the end of use of the vehicle.

7. The information processing apparatus according to claim 6, wherein the controller is configured to control an ignition of the vehicle to remain on until at least first time elapses after the end of use of the vehicle.

8. The information processing apparatus according to claim 5, wherein the controller is configured to control the device to shift into a stop mode at a point in time when second time has elapsed after the shift of the device into the operation mode.

9. The information processing apparatus according to claim 1, wherein the controller is configured to control the window of the vehicle to be open while the vehicle is traveling automatically from a standby location to a dispatch location before the start of use of the vehicle.

10. The information processing apparatus according to claim 1, wherein the controller is configured to control the window of the vehicle to open when the vehicle departs a standby location.

11. The information processing apparatus according to claim 1, wherein the controller is configured to control the window of the vehicle to close before the vehicle arrives at a dispatch location.

12. The information processing apparatus according to claim 1, further comprising a communication interface,
wherein the controller controls the window of the vehicle to open upon receiving from a terminal apparatus of a user, by the communication interface, a dispatch command signal commanding dispatch of the vehicle.

13. The information processing apparatus according to claim 12, wherein the controller controls the window of the vehicle to close upon receiving from the terminal apparatus, by the communication interface, a signal indicating a start of use of the vehicle.

14. The information processing apparatus according to claim 12, wherein the controller controls the vehicle to travel automatically from a standby location to a dispatch location upon receiving, by the communication interface, the dispatch command signal.

15. The information processing apparatus according to claim 1, wherein in a case in which the vehicle waits at a standby location before the start of use of the vehicle, the controller controls the window of the vehicle to be open when an ignition of the vehicle is turned on at the standby location.

16. The information processing apparatus according to claim 1, wherein the controller is configured to determine an amount of opening of the window of the vehicle based on weather information.

17. The information processing apparatus according to claim 1, wherein the controller is configured to control spraying of the cleaning agent and control the window of the vehicle to be open while the vehicle is traveling automatically from a standby location to a dispatch location before the start of use of the vehicle.

18. The information processing apparatus according to claim 1, wherein the cleaning agent includes a deodorizer and/or a sanitizer.

19. A system comprising:
the information processing apparatus according to claim 1; and
the vehicle.

20. A method comprising:
controlling spraying of a cleaning agent in an interior of a vehicle after an end of use of the vehicle; and
controlling a window of the vehicle to be open before a start of use of the vehicle.
